(19) United States
(12) Patent Application Publication
(10) Pub. No.: US 2023/0164514 A1
(43) Pub. Date: May 25, 2023

(54) A METHOD FOR NOTIFYING A FIRST MOBILE TERMINAL OF AN EVENT RELATED TO A SECOND MOBILE TERMINAL

(71) Applicant: Orange, Issy-les-Moulineaux (FR)

(72) Inventors: Shi Jiao, Beijing (CN); Zhihong Guo, Beijing (CN)

(21) Appl. No.: 17/916,784

(22) PCT Filed: Mar. 31, 2021
(86) PCT No.: PCT/IB2021/000212

§ 371 Date: Oct. 3, 2022

(30) Foreign Application Priority Data
Apr. 3, 2020 (CN) PCT/CN2020/083254

(51) Int. Cl. H04W 4/02; H04W 4/80; G16H 50/80

(52) U.S. Cl. CPC H04W 4/023; H04W 4/80; G16H 50/80

(57) ABSTRACT

A method for notifying on a first mobile terminal an event related to a second mobile terminal is disclosed. The method includes collecting first parameters associated with a short-range radio communication device detected by the first mobile terminal, playing information on the first mobile terminal about an event related to the second mobile terminal when a match is detected between the first parameters and second parameters associated with a short-range radio communication device detected by the second mobile device, the second parameters being collected by the second mobile device. A corresponding mobile terminal capable of notifying an event related to another mobile terminal, as well as to a corresponding tracking device is also disclosed. In case of a pandemic, the method informs a user of mobile terminal that (s)he has been in contact with an infected user of another mobile terminal in the recent past.

### CROSS-REFERENCE TO RELATED APPLICATIONS

This application is filed under 35 U.S.C. § 371 as the U.S. National Phase of Application No. PCT/IB2021/000212 entitled “A METHOD FOR NOTIFYING A FIRST MOBILE TERMINAL OF AN EVENT RELATED TO A SECOND MOBILE TERMINAL” and filed Mar. 31, 2021, and which claims priority to PCT/CN2020/083254 filed Apr. 3, 2020, each of which is incorporated by reference in its entirety.

### BACKGROUND

### Field

The present disclosure relates to a method for tracking mobile terminals in order to notify them of the occurrence of an event based on their moving about history.

### Description of Related Technology

In some situations, it would be useful to be capable of contacting nonspecific individuals who may have been in nearby contact with one or more specific individuals in the recent past, in order to contact these nonspecific individuals when necessary.

For instance, in situation of as a pandemic, it would be useful to be capable of contacting nonspecific individuals who may have been in nearby contact in the recent past with one or more individuals infected by a contagious virus, in order to alert these nonspecific individuals that they may have been infected by this virus, and thus allow them to take the necessary measures to prevent further spreading of the disease.

Another example is the situation where one looks for a criminal: it would be useful to be capable of contacting nonspecific individuals who may have been in nearby contact with a criminal suspect in the recent past, in order to request these nonspecific individuals to provide any suspicious event they would have witnessed.

While telecom operators can provide some data making it possible to retrace the route of an individual by means of connection information from the individual's mobile terminal to the various base stations of a cellular network, such information is not always sufficient to retrace precisely an individual's route, in particular when it takes place inside buildings or in underground structures such as metro tunnels, etc. As such, these data do not allow checking whether different individuals have been at the same time in the very same place.

### SUMMARY

The present development was designed with the foregoing in mind.

The present development concerns a method for notifying on a first mobile terminal an event related to a second mobile terminal, comprising:
collecting first parameters associated with at least one short-range radio communication device detected by the first mobile terminal, and
playing information on the first mobile terminal about an event related to the second mobile terminal when a match is detected between at least one of said first parameters and at least one second parameters associated with at least one short-range radio communication device detected by the second mobile device, said second parameters being collected by the second mobile device.

The present solution relies on the use of short-range radio communication techniques such as Wi-Fi, Bluetooth, NFC (Near-Field Communication), etc. which are largely used indoor to provide a better connectivity. Due to the short-range of these communication techniques, the route of an individual can be obtained more precisely than with methods of the prior art.

Thus, throughout its route, a mobile terminal collects parameters associated with the various short-range radio communication devices within the range of which it finds itself. Such short-range radio communication devices may be other mobile terminals, Wi-Fi hotspots, NFC enabled terminals used to control transport tickets, etc.

Knowing the route of a given individual based on these collected parameters, it is possible to inform this individual that he/she encountered an infected individual.

A notification is played on the mobile device of the individual informing him/her of this encounter. This notification can be a message, such as a text message, displayed on a screen of the mobile device, a vocal message played by a speaker of the mobile device, or both.

According to an embodiment of the method for notifying an event related to another mobile terminal, said parameters associated with said at least one short-range radio communication device comprise an identifier of said at least one short-range radio communication device.

Sometimes, an identifier of a short-range radio communication device conveys enough information for a user of a mobile terminal to know the time and place at which he/she may have encountered an infected individual. An example of such an explicit identifier of a short-range radio communication device can be “bestcoffee-shop-toureiffel”. The user of the mobile terminal being notified the identifier of the short-range radio communication device can determine the place of the encounter and a date and time at which he/she stopped at this coffee shop.

According to an embodiment of the method for notifying an event related to another mobile terminal, the parameters associated with said at least one short-range radio communication device further comprise at least one of:
a timestamp indicating a time at which said mobile terminal enters the range of said at least one short-range radio communication device, and
a timestamp indicating a time at which said mobile terminal leaves the range of said at least one short-range radio communication device.

It is thus possible to determine the time at which the encounter took place. This information can help the individual identify people he/she met after the encounter with an affected individual took place and inform them. It is also possible to determine an estimate of the time spent in the vicinity of an infected individual.

According to an embodiment of the method for notifying an event related to another mobile terminal, the collected parameters associated with said at least one short-range radio communication device further comprise a location of said at least one short-range radio communication device or the location of the second mobile terminal when the collected parameters do not comprise the location of said short-range radio communication device.

As a result, an accurate location of the encountered can be notified to the user of the mobile terminal.

According to an embodiment of the method for notifying an event related to another mobile terminal, the method further comprises storing said first parameters in a memory of said first mobile terminal. The collected parameters are then stored locally, either for being processed by the mobile device or to be transmitted later on to a remote processing device.

According to an embodiment of the method for notifying an event related to another mobile terminal, the first parameters are encrypted prior to their storing. The collected parameters are then protected in case the mobile terminal get hacked.

According to an embodiment of the method for notifying an event related to
another mobile terminal, when the collected parameters are processed by the mobile terminal, the method further comprises:
receiving said second parameters associated with at least one short-range radio communication device detected by the second mobile device,
determining a match between at least one first parameters and at least one second parameters associated with at least one short-range radio communication device detected by the second mobile device, the determination of a match between at least one first parameter and at least one second parameter triggering the playing of information on the first mobile terminal about an event related to the second mobile terminal.

In this implementation, the mobile terminal receives parameters associated with at least one short-range radio communication device collected by a mobile terminal whose user is infected. The parameters associated with said short-range radio communication device may comprise the location of said short-range radio communication device, a time and a location.

By comparing this information of time and location, the mobile device determines if there is a match, i.e. was the mobile terminal within range of the short-range radio communication device of the infected individual.

If there is a match, a notification is played on the mobile device informing the user of this encounter.

This implementation is more protective, in terms of user's privacy: the user device history is uploaded to a remote server only when necessary, i.e. when there is a request from other users who wish to know if they were in the vicinity of the infected individual.

According to an embodiment of the method for notifying an event related to another mobile terminal, the method further comprises:

transmitting said first parameters to a remote server storing said second parameters,

receiving said information about an event related to the second mobile terminal when said remote server detects a match between at least one first parameters and at least one second parameters.

By comparing this information of time and location, the remote server determines if there is a match, i.e. was the mobile terminal within range of the short-range radio communication device of the infected individual.

If there is a match, a request for playing a notification on the mobile device informing the user of this encounter is sent to the mobile device.

This implementation requires a large data storage capacity on the remote server, but then the inquiry to find individuals in order to inform them can be made directly on the remote server, in a transparent way for the mobile terminal itself.

Another object of the development is a method for tracking a mobile terminal, said method being executed by a tracking device and comprising:
receiving first parameters associated with at least one short-range radio communication device detected by a first mobile terminal and collected by said first mobile terminal,
receiving second parameters associated with at least one short-range radio communication device detected by a second mobile terminal and collected by said second mobile terminal,
determining if there is a match between at least one first parameters and at least one second parameters, and
when there is a match between said at least one first parameters and said at least one second parameters, requesting a playing of information on the first mobile terminal about an event related to the second mobile terminal.

The development also concerns a mobile terminal capable of notifying an event related to another mobile terminal, said mobile terminal comprising a processor configured to:
collect first parameters associated with at least one short-range radio communication device detected by the mobile terminal, and
play information on the mobile terminal about an event related to the other mobile terminal when a match is detected between at least one first parameters and at least one second parameters associated with at least one short-range radio communication device detected by the other mobile device, said second parameters being collected by the other mobile device.

A fourth object of the development is a tracking device capable of tracking a mobile terminal, said a tracking device comprising a processor configured to:
receive first parameters associated with at least one short-range radio communication device detected by a first mobile terminal and collected by said first mobile terminal,
receive second parameters associated with at least one short-range radio communication device detected by a second mobile terminal and collected by said second mobile terminal,
determine if there is a match between at least one first parameters and at least one second parameters, and
when there is a match between said at least one first parameters and said at least one second parameters, request a playing of information on the first mobile terminal about an event related to the second mobile terminal.

According to an embodiment of the development, said tracking device is embedded in the mobile device as previously discussed.

According to another embodiment of the development, said tracking device is embedded in a remote server.

Some processes implemented by elements of the development may be computer implemented. Accordingly, such elements may take the form of an entirely hardware embodiment, an entirely software embodiment (including firmware, resident software, micro-code, etc.) or an embodiment combining software and hardware aspects that may all generally be referred to herein as a “circuit”, “module” or “system”. Furthermore, such elements may take the form of a computer program product embodied in any tangible medium of expression having computer usable program code embodied in the medium.

Since elements of the present development can be implemented in software, the present development can be embodied as computer readable code for provision to a programmable apparatus on any suitable carrier medium. A tangible carrier medium may comprise a storage medium such as a floppy disk, a CD-ROM, a hard disk drive, a magnetic tape device or a solid-state memory device and the like. A transient carrier medium may include a signal such as an electrical signal, an electronic signal, an optical signal, an acoustic signal, a magnetic signal or an electromagnetic signal, e.g. a microwave or RF signal.

### BRIEF DESCRIPTION OF THE DRAWINGS

The present disclosure can be better understood with reference to the following description and drawings, given by way of example and not limiting the scope of protection, and in which:

FIG. 1 represents a system in which different embodiments of the method for notifying an event and the method for tracking a mobile terminal can be executed,

FIG. 2 represents a diagram of exchanges between different communication devices involved in the implementation of the method for notifying an event and the method for tracking a mobile terminal in a first embodiment of the development,

FIG. 3 represents a diagram of exchanges between different communication devices involved in the implementation of the method for notifying an event and the method for tracking a mobile terminal in a second embodiment of the development,

FIG. 4 represents a detailed view of one of the mobile terminal 10 according to the development, and

FIG. 5 represents a detailed view of the tracking device 15 according to the development.

The components in the figures are not necessarily to scale, emphasis instead being placed upon illustrating the principles of the development.

### DETAILED DESCRIPTION OF CERTAIN ILLUSTRATIVE EMBODIMENTS

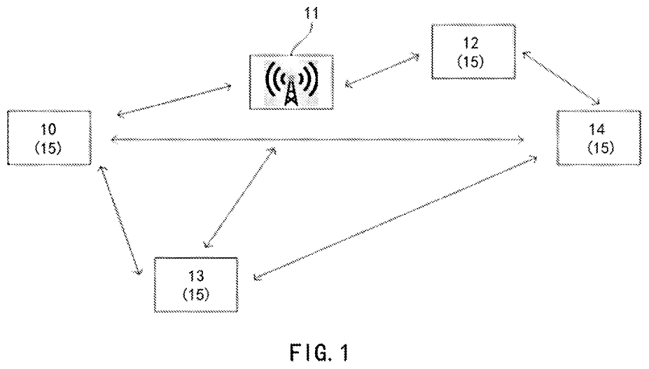

FIG. 1 represents a system in which different embodiments of the method for notifying an event and the method for tracking a mobile terminal can be executed.

The system comprises at least a first mobile terminal 10 and a second mobile terminal 12, a first short-range radio communication device 11 (which in this example is a Wi-Fi access node), a second short-range radio communication devices 13 (which in this example is another mobile terminal) and a remote server 14.

In the following, the second mobile terminal 12 is supposed to be owned by a specific individual, such as an individual being diagnosed as infected by a contagious virus. Mobile terminals 10 and 12, short-range radio communication device 11, 13 and remote server 14 may embed a tracking device 15, as discussed thereafter.

The mobile terminals 10, 12 may each possess a respective unique identifier Id10, Id12 such as a universally unique identifier (UUID) defined in the document RFC 4122 published by the IETF (Internet Engineering Task Force). A UUID is a 128-bit number used to identify information in computer systems.

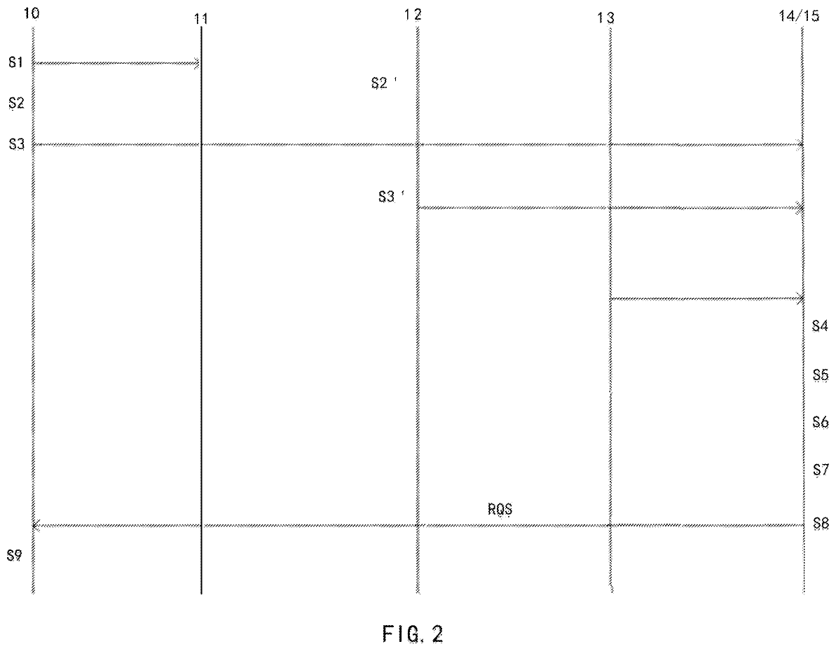

The functions and interconnexions between those different devices will be explained in reference to FIGS. 2-5.

FIG. 2 represents a diagram of exchanges between different communication devices involved in the implementation of the method for notifying an event and the method for tracking a mobile terminal in a first embodiment of the development. In this first implementation, only the remote server 14 embeds a tracking device 15.

In step SI, the mobile terminal 10 enters the range of the first short-range radio communication device 11. This is typically the case when the mobile terminal 10 starts detecting a radio signal constantly or periodically emitted by device 11.

In a step S2, the mobile terminal 10 collects parameters associated with the short-range radio communication device 11 it has detected. These parameters comprise a unique identifier Id11 of this short-range radio communication device 11, here the SSID of the Wi-Fi network provided by the short-range radio communication device 11 when device 11 is a Wi-Fi access node.

These collected parameters may optionally comprise as well a location of the short-range radio communication device 11, if provided. In a specific embodiment where the mobile terminal 10 fails to obtain this location of the short-range radio communication device 11, the mobile terminal 10 may instead collect its own location, obtained for instance by means of a geo-localization module (e.g. GPS) embedded in the mobile terminal 10.

Once collected, those parameters are timestamped, for instance to associate a specific day and time with the identifier of the detected device 11. In order to do so, for each detected short-range communication device 11, 13, the collected parameters may comprise the identifier Id11, Id13 in association with:
a timestamp indicating a time at which mobile terminal 10 has entered the range of, or connected with, this short-range radio communication,
a timestamp indicating a time at which mobile terminal 10 has left the range of, or disconnected from, this short-range radio communication device (i.e. the time at which it does not detect it any longer), or
both these above-mentioned time stamps.

In a first embodiment, step S2 is executed when the mobile terminal 10 connects to the short-range radio communication device 11. In this embodiment where only the connection to a short-range radio communication device triggers the parameters collection, there can be holes in time, in terms of user localization, since the mobile terminal 10 does not continuously connect to a short-range radio communication device such as devices 11 or 13.

In another embodiment, the mobile terminal 10 collects parameters associated with any short-range radio communication device 11, 13 it detects, without actually establishing a connection with these short-range radio communication device 11,13. In this embodiment, the mobile terminal 10 collects parameters associated with each the short-range radio communication device 11, 13 it passes by, as long as the mobile terminal 10 passes within their range.

In this embodiment, not only the mobile terminal 10 collects parameters when connecting to short-range radio communication device 11, but also when detecting a nearby short-range radio communication device 13, without connecting to it. As a consequence, this embodiment gives a more precise picture of the mobile terminal 10 route.

From time to time, the mobile terminal 10 transmits the collected parameters of each short-range radio communication device 11, 13 it has encountered, for example as a list L10, to the remote server 14. This transmission occurs during a step S3.

More precisely, for each short-range radio communication device 11, 13 encountered, the mobile terminal 10 stores in list L10 the respective identifier Id11, Id13 of the short-range radio communication devices 11,13 (e.g. the SSIDs of the Wi-Fi network provided by the short-range radio communication devices 11 and 13) or any other identifier of a short-range radio connection provided by these short-range radio communication devices 11, 13, with also possibly a respective location of these short-range radio communication devices 11,13 and at least a timestamp indicating a time at which those parameters are collected.

In case a connection is established between the mobile terminal 10 and a short-range radio communication device 11 or 13, a duration of this connection may be stored in the list L10 as well.

Steps S′2 and S′3, similar respectively to steps S2 and S3 as above, are also executed by mobile terminal 12.

In a step S4, the remote server 14 receives a message about an event related to terminal mobile 12 which should be notified to other mobile terminals. Such an event may be for instance the fact that a user of mobile this mobile terminal 12 has been diagnosed as being infected by a contagious virus. A possible date of infection is also provided to the remote server 14. The infection of the user of the mobile terminal 12 may be thus referred to as an event related to this mobile terminal 12. The remote server 14 forwards this information to the tracking device 15.

For each mobile terminal different from the mobile terminal 12 related to the event to notify (here for mobile terminal 10 in FIG. 2, but could be for any greater number of mobile terminals), the tracking device 15 uses the parameters collected by this mobile terminal 10 and the parameters collected by the mobile terminal 12, in order to find individuals which have been in the vicinity of the user of the mobile terminal 12, at a given time. Here, when also provided, the supposed date of infection may be used to determine a time window in which the server 14 will look for individuals which have been in the vicinity of the user of mobile terminal 12. This time window can take into account the incubation period of the disease.

In a step S5, the tracking device 15 extracts from list L10 a first set of parameters collected by mobile terminal 10 and whose timestamps are within the time window. The tracking device 15 extracts as well, from a list L12, a second set of parameters collected by mobile terminal 12 and whose timestamps are within the time window.

In a step S6, the tracking device 15 compares the first set of parameters collected by mobile terminal 10 with the second set of parameters collected by mobile terminal 12 in order to determine if there is at least one parameter related to the same short-range radio communication device 11 or 13 in both first and second set of parameters.

In that respect, when an identifier Id11 or Id13 of a short-range radio communication device 11, 13 has been collected in both the first and the second set of parameters, with the same associated timestamps or relatively close timestamps (for instance within an hour interval), then it is determined that the mobile terminal 10 was in the vicinity of the mobile terminal 12 of the infected individual substantially at the same time during the time window.

In a specific embodiments where the collected parameters comprise both time stamps indicating when the mobile terminal has entered into the range (resp. has connected with) and has left the range (resp. has disconnected with) of the short-range communication device, then a verification is made as to whether the same identifier Id11 or Id13 is collected by both terminals with overlapping time intervals (defined by the collected time stamps). When this is the case, then it is determined that the mobile terminal 10 was in the vicinity of the mobile terminal 12 of the infected individual substantially at the same time during the time window.

In another specific embodiment where the tracking device 15 knows about the identifier Id12 of the mobile terminal acting as a short-range radio communication device (i.e. when it acts as a wifi access point for instance), if the tracking device 15 determines that this identifier Id12 of the mobile terminal 12 is comprised in the first set of parameters collected by mobile terminal 10 whose timestamps are within the time window, then the tracking device 15 may determine that the mobile terminal 10 was within direct short range of the mobile terminal 12 of the infected individual at some time during the time window.

Once a match between the mobile terminal 10 and the mobile terminal 12 of the infected user is detected, the tracking device 15 embedded in the remote server 14 generates, m a step S7, one or more parameters associated with said event. These parameters comprise information that an infected individual was in the vicinity mobile terminal 10. They may also comprise identifiers Id11, Id13 of the short-range radio communication device 11, 13 identified by the tracking device 15, the location of said short-range radio communication device 11, 13, and an indication of a time and date at which the encounter occurred.

In a step S8, the tracking device 15 embedded in the remote server 14 sends a request RQS to the mobile terminal 10 comprising the parameters associated with the event and requesting the mobile terminal 10 to play a notification comprising information about said event, such as “you were near an infected individual”, for instance.

When additional information such as the location of the short-range communication device (or of the mobile terminal when it has failed to collect it) or a timestamp associated with the identifier Id11, Id13 has been collected, this information can be used to enrich the notification, such as “you were near an infected individual on Month Day Year, time, at location X” for instance.

In a step S9, the mobile terminal 10 plays this notification. This notification can be a message, such as a text message, displayed on a screen of the mobile device 10, or a vocal message played by a speaker of the mobile device 10, or both.

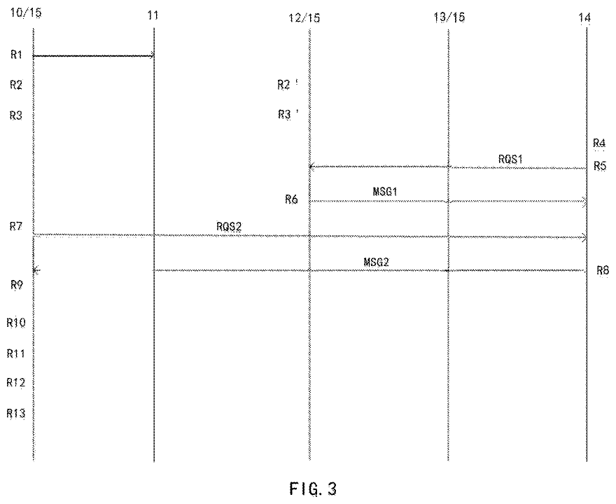

FIG. 3 represents a diagram of exchanges between different communication devices involved in the implementation of the method for notifying an event and the method for tracking a mobile terminal in a second embodiment of the development. In this second implementation, the tracking device 15 is embedded in mobile terminal 10, and in short-range radio communication devices 12 and 13.

In a step RI, the mobile terminal 10 enters the range of the first short-range radio communication device 11.

In a step R2, the mobile terminal 10 collects parameters associated with the short-range radio communication device 11, including its unique identifier Id11, here the SSID of the Wi-Fi network provided by the short-range radio communication device 11 since device 11 is a Wi-Fi access node. The collected parameters may also possibly comprise a location of the short-range radio communication device 11 or, when the mobile terminal fails to obtain such a location, the location of the mobile terminal 10 itself, as described previously. Once collected, those parameters are timestamped as described in relation to FIG. 2.

Just as in the previous embodiment, step R2 may be executed when the mobile terminal 10 connects to a short-range radio communication device 11,13 or when it detects one of them, without establishing a connection with the short-range radio communication device 11, 13.

In a step R3, the mobile terminal 10 stores locally the collected parameters of each short-range radio communication device 11, 13, it has encountered, for example as a list L10, in its memory. To increase data privacy, the list L10 is encrypted prior to being stored.

For each short-range radio communication device 11, 13 encountered, the mobile terminal 10 stores in list L10 the identifier Id11, Id13, of the short-range radio communication devices 11, 13 (e.g. the SSID of the Wi-Fi connection provided by the short-range radio communication devices 11) or any other identifier of a short-range radio connection provided by the short-range radio communication device 13, with possibly a location of the short-range radio communication devices 11, 13 (or alternatively of the mobile terminal 10 itself when it fails to obtain it), and at least a timestamp indicating a time at which those parameters are collected. In case a connection is established between the mobile terminal 10 and one of short-range radio communication devices 11, 13, a duration of this connection may also be stored in the list L10 as well.

Steps R2′ and R3′, which are similar respectively to steps R2 and R3 as above, are also executed by mobile terminal 12.

At this stage, each one of mobile terminals 10 and 12 stores a list comprising information about its whereabouts.

In this second implementation, there is no automatic upload of all the collected parameters to the remote server 14, but rather an upload of parameters collected by a specific mobile terminal 10 or 12, when necessary.

In a step R4, the remote server 14 receives a message informing it that a user of mobile terminal 12 is, for example, infected by a contagious disease. A possible date of infection may also be provided to the remote server 14 at this stage, this date of infection being useable to determine a time window in which individuals who have been in the vicinity of the user of mobile terminal 12 will be looked for. This time window can take into account the incubation period of the disease. The infection of the user of mobile terminal 12 is referred to as an event related to this mobile terminal 12.

In a step R5, the remote server 14 sends a request RQS1 to the mobile terminal 12, the user of which is infected, asking it to transmit its list L12 of collected parameters to the remote server 14 where it is stored together with the identifier Id12 of the mobile terminal 12, for later use.

In a step R6, the mobile terminal 12 transmits its list L12 of collected parameters to the remote server 14 together with its identifier Id12 in a message MSG1.

At some point, a user of the mobile terminal 10 wants to know if he/she was in contact with an infected individual. The user of the mobile terminal 10, by means of a user interface of the mobile terminal 10, triggers the sending of a request RQS2 to the remote server 14, in a step R7, asking for the lists of collected parameters of mobile terminals whose user declared themselves as infected.

In a step R8, in response to request RQS2, the remote server 14 transmits the list L12 of parameters collected by the mobile terminal 12, together with the supposed date of infection of the user of mobile terminal 12 and/or the time window, to the mobile terminal 10 in a message MSG2. Here, the list L12 collected by only one mobile terminal 12 is transmitted for illustration, but several lists, respectively collected by different mobile terminals belonging to infected users, could be transmitted to mobile terminal 10, naturally.

In a step R9, the tracking device 15 embedded in mobile terminal 10 uses the parameters collected by mobile terminal 10 and the parameters collected by mobile terminal 12, together with the time window, in order to determine if the mobile device 10 and the mobile terminal 12 have been in the vicinity of the same short-range radio communication device 11, 13 around the same given time.

In a step R10, the tracking device 15 extracts from list L10 a first set of parameters collected by mobile terminal 10 and whose timestamps are within the time window. The tracking device 15 extracts as well, from a list L12 a second set of parameters collected by mobile terminal device 12 and whose timestamps are within the time window.

In a step R11, the tracking device 15 compares the first set of parameters collected by mobile terminal 10 with the second set of parameters collected by mobile terminal 12 in order to determine if there is at least one parameter related to a short-range radio communication device 11, 13 which is common to the first and the second set of parameters.

For instance, when an identifier Id11 or Id13 of a short-range radio communication device 11, 13 is common to the first and the second set of parameters, then it is determined that the mobile terminal 10 was in the vicinity of the mobile terminal 12 of the infected individual at some time during the time window.

Once a match between the mobile terminal 10 and mobile terminal 12 of the infected user is detected, the tracking device 15 embedded in the mobile terminal 4 generates, in a step R12, one or more parameters associated with said event. These parameters comprise an information that an infected individual was in the vicinity mobile terminal 10. They may also comprise an identifier Id11, Id13 of the short-range radio communication device 11, 13 identified by the tracking device 15, the location of said short-range radio communication device 11, 13, and an indication of a time and date at which the encounter occurred.

In a step R13, the tracking device 15 triggers the playing a notification comprising information about said event such as “you were near an infected individual on Month Day Year, time, at location X”. This notification can be a message, such as a text message, displayed on a screen of the mobile device 10, or a vocal message played by a speaker of the mobile device 10, or both.

In another embodiment of the second implementation of the method for notifying an event and the method for tracking a mobile terminal, the tracking device 15 is embedded in the remote server 14, not in the mobile terminal 10, and steps R8 to R13 are not executed. Instead, in this embodiment, a user of the mobile terminal 10 who wants to know if he/she was in contact with an infected individual, triggers, by means of the user interface of the mobile terminal 10, the transmission of its own list L10 of collected parameters to the remote server 14 together with a request for determining whether the mobile terminal 10 was within range of a short-range radio communication device 12 belonging to an infected individual or not.

Upon reception of this request by the remote server 14, steps S5 to S9 are executed as explained in reference to FIG. 2.

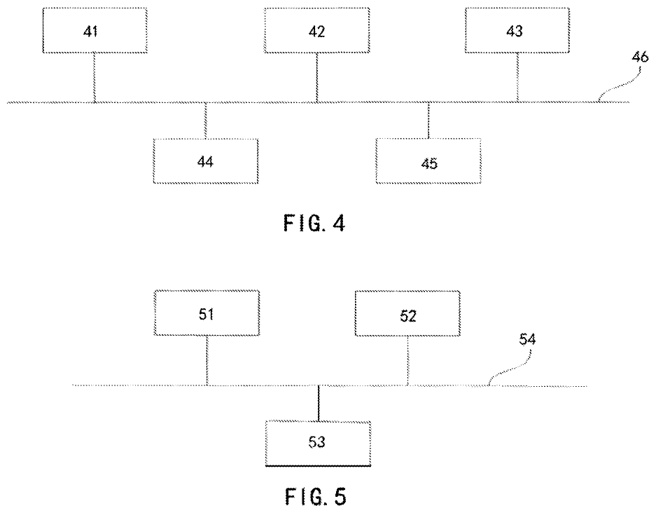

FIG. 4 represents a detailed view of one of the mobile terminal 10 according to the development.

A mobile terminal 10 may comprise at least one hardware processor 41, a storage unit 42, an input device 43, a display device 44, and a network interface 45, which are typically connected by a data bus 46. Of course, constituent elements of the mobile terminal 10 may be connected by a connection other than a data bus connection.

The hardware processor(s) 41 controls operations of the mobile terminal 10. The storage unit 42 stores at least one program capable of notifying a mobile terminal of an event linked to another mobile terminal to be executed by the processor 41, and various data, such as parameters used by computations performed by the processor 41, intermediate data of computations performed by the processor 41, and so on. The processor 41 may be formed by any known and suitable hardware, or software, or a combination of hardware and software. For example, the processor 41 may be formed by dedicated hardware such as a processing circuit, or by a programmable processing unit such as a CPU (Central Processing Unit) that executes a program stored in a memory thereof

The storage unit 42 may be formed by any suitable storage or means capable of storing the program, data, or the like in a computer-readable manner. Examples of the storage unit 42 include non-transitory computer-readable storage media such as semiconductor memory devices, and magnetic, optical, or magneto-optical recording media loaded into a read and write unit. The program causes the processor 41 to perform a process according to an embodiment of the present development as described with reference to FIGS. 2 and 3.

The input device 43 may be formed by a keyboard, a pointing device such as a mouse, or the like for use by the user to input commands, for example to make user's selections of parameters used for selecting the level of proficiency of the reader in reading logograms.

The display device 44 and the input device 43 may be formed integrally by a touchscreen panel.

A network interface 45 provides a connection between the mobile terminal 10 and short-range radio communication devices 11, 12, 13 and a remote server 14.

FIG. 5 represents a detailed view of the tracking device 15 according to the development.

A tracking device 15 may comprise at least one hardware processor 51, a storage unit 52, and an interface 53, which are typically connected by a data bus 54. Of course, constituent elements of the tracking device 15 may be connected by a connection other than a data bus connection.

The hardware processor(s) 51 controls operations of the tracking device 15. The storage unit 52 stores at least one program capable of tracking a mobile terminal to be executed by the processor 51, and various data, such as parameters used by computations performed by the processor 51, intermediate data of computations performed by the processor 51, and so on. The processor 51 may be formed by any known and suitable hardware, or software, or a combination of hardware and software. For example, the processor 51 may be formed by dedicated hardware such as a processing circuit, or by a programmable processing unit such as a CPU (Central Processing Unit) that executes a program stored in a memory thereof

The storage unit 52 may be formed by any suitable storage or means capable of storing the program, data, or the like in a computer-readable manner. Examples of the storage unit 52 include non-transitory computer-readable storage media such as semiconductor memory devices, and magnetic, optical, or magneto-optical recording media loaded into a read and write unit. The program causes the processor 51 to perform a process according to an embodiment of the present development as described with reference to FIGS. 2 and 3.

The interface 53 provides a connection between the tracking device 15 and the mobile terminal 10 or the remote server 14.

Although the present development has been described hereinabove with reference to specific embodiments, the present development is not limited to the specific embodiments, and modifications will be apparent to a skilled person in the art which lie within the scope of the present development.

In particular, the present development has been described in details in the specific context of the notification, on a mobile terminal, that the user of another terminal has been diagnosed as infected by a contagious virus. However, the present development can apply to any other type of event worthy to be notified on a mobile terminal, when this event is related to another mobile terminal that has been in short proximity of this terminal in a recent past.

In addition, the case of a single mobile terminal 10 being notified has been illustrated in FIGS. 2 and 3, for the sake of explaining the development. However, any number of mobile terminals 10 can be notified of an event related to a mobile terminal 12, using the same embodiments as discussed previously. Similarly, the case of a single mobile terminal 12 triggering the notification on mobile terminal 10 has been illustrated in FIGS. 2 and 3, for the sake of explaining the development. However, events related to any number of mobile terminals 12 can be notified on one (or more) mobile terminal 10, using the same embodiments as discussed previously.

Many further modifications and variations will suggest themselves to those versed in the art upon making reference to the foregoing illustrative embodiments, which are given by way of example only and which are not intended to limit the scope of the development, that being determined solely by the appended claims. In particular the different features from different embodiments may be interchanged, where appropriate.

1. A method for notifying on a first mobile terminal an event related to a second mobile terminal, the method comprising:
collecting first parameters associated with at least one short-range radio communication device detected by the first mobile terminal; and
playing information on the first mobile terminal about an event related to the second mobile terminal when a match is detected between at least one of the first parameters and at least one second parameters associated with at least one short-range radio communication device detected by the second mobile device, the second parameters being collected by the second mobile device.

2. The method for notifying on a first mobile terminal an event related to a second mobile terminal according to claim 1, wherein the parameters associated with the at least one short-range radio communication device comprise an identifier of the at least one short-range radio communication device.

3. The method for notifying on a first mobile terminal an event related to a second mobile terminal according to claim 2, wherein the parameters associated with the at least one short-range radio communication device further comprise at least one of:
a timestamp indicating a time at which the mobile terminal enters the range of the at least one short-range radio communication device, and
a timestamp indicating a time at which the mobile terminal leaves the range of the at least one short-range radio communication device.

4. The method for notifying on a first mobile terminal an event related to a second mobile terminal according to claim 2, wherein the collected parameters associated with the at least one short-range radio communication device further comprise a location of the at least one short-range radio communication device or wherein the method further comprises collecting the location of the second mobile terminal when the collected parameters do not comprise the location of the short-range radio communication device, the information played on the first mobile terminal comprising the location.

5. The method for notifying on a first mobile terminal an event related to a second mobile terminal according to claim 1, further comprising storing the first parameters in a memory of the first mobile terminal.

6. The method for notifying on a first mobile terminal an event related to a second mobile terminal according to claim 5, wherein the first parameters are encrypted prior to their storing.

7. The method for notifying on a first mobile terminal an event related to a second mobile terminal according to claim 1, comprising:
receiving the second parameters associated with at least one short-range radio communication device detected by the second mobile device; and
determining a match between at least one first parameters and at least one second parameters associated with at least one short-range radio communication device detected by the second mobile device, the determination of a match between at least one first parameter and at least one second parameter triggering the playing of information on the first mobile terminal about an event related to the second mobile terminal.

8. The method for notifying on a first mobile terminal an event related to a second mobile terminal according to claim 1, further comprising:
transmitting the first parameters to a remote server storing the second parameters; and
receiving the information about an event related to the second mobile terminal when the remote server detects a match between at least one first parameters and at least one second parameters.

9. A method for tracking a mobile terminal, the method being executed by a tracking device and comprising:
receiving first parameters associated with at least one short-range radio communication device detected by a first mobile terminal and collected by the first mobile terminal;
receiving second parameters associated with at least one short-range radio communication device detected by a second mobile terminal and collected by the second mobile terminal;
determining if there is a match between at least one first parameters and at least one second parameters; and
when there is a match between the at least one first parameters and the at least one second parameters, requesting a playing of information on the first mobile terminal about an event related to the second mobile terminal.

10. A mobile terminal capable of notifying an event related to another mobile terminal, the mobile terminal comprising a processor configured to:
collect first parameters associated with at least one short-range radio communication device detected by the mobile terminal; and
play information on the mobile terminal about an event related to the other mobile terminal when a match is detected between at least one of the first parameters and at least one second parameters associated with at least one short-range radio communication device detected by the other mobile terminal, the second parameters being collected by the other mobile terminal.

11. A tracking device capable of tracking a mobile terminal, the tracking device comprising a processor configured to:
receive first parameters associated with at least one short-range radio communication device detected by a first mobile terminal and collected by the first mobile terminal;
receive second parameters associated with at least one short-range radio communication device detected by a second mobile terminal and collected by the second mobile terminal;
determine if there is a match between at least one first parameters and at least one second parameters; and
when there is a match between the at least one first parameters and the at least one second parameters, request a playing of information on the first mobile terminal about an event related to the second mobile terminal.

12. The tracking device according to claim 11, wherein the tracking device is embedded in the first mobile terminal.

13. The tracking device according to claim 11, wherein the tracking device is embedded in a remote server.

14. A processing circuit comprising a processor and a memory, the memory storing program code instructions of a computer program for the implementation of the method for notifying an event according to claim 1 when the computer program is executed by the processor.

15. A processing circuit comprising a processor and a memory, the memory storing program code instructions of a computer program for the implementation of the method for tracking a mobile terminal according to claim 9 when the computer program is executed by the processor.
